(19) United States
(12) Patent Application Publication
(10) Pub. No.: US 2007/0281044 A1
(43) Pub. Date: Dec. 6, 2007

(54) USE OF COLD-PRESSED EDIBLE SEED OILS, FLOURS, AND/OR BLENDS THEREOF AS ANTI-INFLAMMATORY AND COX-2 INHIBITORY AGENTS

(76) Inventors: Mark J. Mueller, Spooner, WI (US); Liangli Yu, Ellicott City, MD (US)

Correspondence Address: BRIGGS AND MORGAN P.A., 2200 IDS CENTER, 80 SOUTH 8TH ST, MINNEAPOLIS, MN, 55402, US

(21) Appl. No.: 11/421,926

(22) Filed: Jun. 2, 2006

(51) Int. Cl. A61K 36/23; A61K 36/889; A61K 36/45; A61K 36/73; A61K 36/87

(52) U.S. Cl. 424/727; 424/732; 424/765; 424/766; 424/776

(57) ABSTRACT

The present invention relates to an anti-inflammatory and COX-2 inhibitory composition that contains at least one of cold-pressed edible seed oils, flours and/or their derivatives having anti-inflammatory and COX-2 inhibitory activity as an active ingredient. The seed oils can be derived from at least one of red raspberry, black raspberry seed, black cumin seed, caneberry seed, raspberry seed, blackberry seed, marionberry seed, boysenberry seed, evergreen blackberry seed, coriander seed, sea buckthorn seed, palm fruit seed, cardomon seed, evening primrose seed, cranberry seed, mullein seed, parsley seed, carrot seed, blueberry seed, grape seed, chardonnay grape seed or merlot grape seed.

### FIELD OF THE INVENTION

The present invention relates to the use of cold-pressed edible seed oils, flours, and/or their derivatives as anti-inflammatory and COX-2 inhibitory agents. The present invention also relates to the anti-inflammatory preparations containing the cold-pressed edible oils, flours and their derivatives, as well as the procedure to produce the anti-inflammatory and COX-2 inhibitory preparations. The derivatives include the blends of oils and flours in their original form with or without additional bulking agents such as starch. The derivatives also included the oils and flours treated by enzymes, acids, extrusion, encapsulation, coating, or a combination of more than one of these treatments.

### BACKGROUND OF THE INVENTION

Cyclooxygenases (COX) catalyze the formation of endoperoxide H₂ from arachidonic acid, and is an important enzyme involved in prostanoid biosynthesis. The prostanoids are lipidic mediators involved in both physiological and pathological process such as inflammation and cancer (Leval et al., 2004). It is known that there are two COX isoforms, COX-1 and COX-2. COX-2 is mainly expressed during pathological processes, whereas COX-1 is expressed ubiquitously. COX-2 inhibition is a possible way to screen for novel potential anti-inflammatory agents (Michaux and Charlier, 2004).

COX-2 inhibitors may also have cancer preventive effects. Overexpression of COX-2 and increased prostaglandin biosynthesis are correlated with carcinogenesis and metastasis at most anatomic sites (Harris et al., 2005; Swamy et al., 2004). Regular intake of COX-2 inhibitors may reduce risk of cancer (Patel et al., 2005).

It is an object of the present invention to provide preparations which are COX-2 inhibitors and which can be used as anti-inflammatory agents.

### SUMMARY OF THE INVENTION

The present invention relates to a method of treating inflammation or inhibiting COX-2 activity in a subject by the administration of effective dosage amounts of preparations comprising cold-pressed edible oils, flours and/or derivatives thereof. A further embodiment of this invention relates to making anti-inflammatory and COX-2 inhibitory preparations comprising cold-pressed edible oils, flours and/or derivatives thereof. Derivatives comprise blends of such oils and flours in their original form with or without acceptable excipient, carriers and/or bulking agents. Derivatives may also comprise oils and/or flours which have been treated by enzymes, acids, extrusions, encapsulation, coating, or a combination of one or more of these treatments.

### BRIEF DESCRIPTION OF THE FIGURES

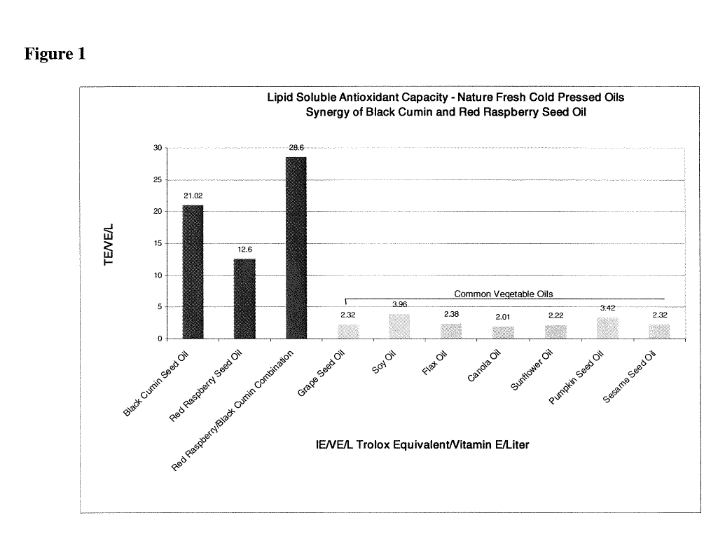

The black cumin and red raspberry seed oil antioxidant synergy it shown in FIG. 1.

### HIGH ANTIOXIDANT COLD PRESSED PLANT OILS

Natural oils derived from plant materials are produced through a method of cold pressing the raw plant material. In a preferred embodiment red raspberry seeds, Rubus ideaus L. and or Black Cumin seeds, nigella sativa, also known as black seed or black caraway oil are used. Black cumin is an agricultural crop in the middle east, India and Asia.

Red raspberry pulp fiber including seeds are recovered from a fruit juice or fruit concentrate food processing operation and dried to a moisture content of less than 10 percent in a low temperature dryer having rapid air flow and with temperatures not exceeding 120 degrees Fahrenheit. Rapid drying of the seed and pulp material is important in the prevention of growth of microorganisms and prevention of decay. Following drying, the seeds are separated from the pulp using a seed cleaner.

The seeds either black cumin or red raspberry are then carefully pressed in a cold press where temperatures of the extracted oil do not exceed 120 degrees Fahrenheit. A Komet™ single or double screw expeller, Model DD85, manufactured by IBG Montforts GmbH was used. Other cold presses are commercially available and could also satisfactorily be used to extract the oil. Typically the press achieves pressures of 1200 pounds per square inch against the seed material and press head. The press may be electrically driven or driven by other mechanical means. The press cylinder, where the oil exits the press, may be enclosed within a hood or shield and put under pressure of an inert gas such as nitrogen or carbon dioxide to prevent the freshly expelled oil from contact with oxygen to enhance the oxidative stability of the oil. To start the process no external heat is used. This is unlike other conventional pressing methods for grains and vegetable oils which add heat to the pressing head. Using a speed of 20 to 100 rpm of the Komet™ press, the oil is extracted at ambient temperatures of 70 to 90 degrees Fahrenheit. A nozzle at the press head having a round aperture ranging from 6 mm to 15 mm allows the solid seed material to be expelled from the press. The oil flows by gravity to a collection container. In one embodiment the fresh, carefully expelled oil is allowed to settle to the bottom of the container and the oil clarifies. In another embodiment the oil is collected and used in an unsettled unrefined condition. A filter can also be used for separation of the fine fiber material from the oil. The oil is then decanted for further storage and bottling.

The solid seed material, sometimes called seed meal or seed press cake can be milled to small particle size using conventional milling equipment such as a hammer mill or roller mill. Typical milling sizes range from 40 screen mesh to 120 screen mesh, but can be produced to even smaller sizes if needed.

The resulting oils of red raspberry and black cumin are very high in antioxidants in comparison to common vegetable oils such as soy oil, canola oil and flax seed oil as can be seen in Table 1, Lipid Soluble Antioxidant Capacity of Cold Press Oils. Further, when black cumin oil is combined with red raspberry seed oil a synergy is created whereby the total antioxidant value of the composition is greater than the arithmetic mean value of the individual components. The Black Cumin and Red Raspberry Seed Oil Antioxidant Synergy is illustrated in both Table 1 and FIG. 1.

TABLE 1
Lipid Soluble Antioxidant Capacity - Cold Pressed Oils
And Synergy of Black Cumin and Red Raspberry Seed Oil

| | Trolox Equivalents/ |
|---|---|
| | Vitamin E/Litre |
| Flax Oil | 2.38 |
| Canola Oil | 4.11 |
| Sesame Seed Oil | 2.32 |
| Sunflower Oil | 2.22 |
| Safflower Oil | 3.11 |
| Soy Oil | 3.96 |
| Red Raspberry Seed Oil | 12.63 |
| Black Cumin Oil | 21.02 |
| Black Cumin/Red Raspberry Combination | 28.57 |

TABLE 2
Fatty Acid Composition of Red Raspberry Seed Oil

| Fatty Acid | grams/100 g |
|---|---|
| Myristic | Nd |
| Palmitic | 1.2–1.6 |
| Palmitoleic | Nd |
| Stearic | 0.1 |
| Oleic | 6.1–7.7 |
| Linoleic | 55.8–57.6 |
| Linolenic | 29.7 |
| Arachidonic | Nd |
| Total Sat | 1.2–1.6 |
| Total MUFA | 6.1–7.7 |
| Total \acute{ω}-3 FA | 35.2 |
| Total PUFA | 91.1–93.0 |

nd = not detectable.
MIJFA and PUFA represent mono and polyunsaturated fatty acids. \acute{ω}-3 FA + \acute{ω}-3 fatty acids.

TABLE 3
Fatty Acid Composition of Black Cumin Seed Oil

| Fatty Acid | grams/100 g |
|---|---|
| Myristic | .24 |
| Palmitic | 12.1 |
| Palmitoleic | .97 |
| Stearic | 3.4 |
| Oleic | 24.0 |
| Linoleic | 55.8 |
| Linolenic | .24 |
| Arachidonic | .21 |
| Total Sat | 13.8 |
| Total MUFA | 25.4 |
| Total \acute{ω}-3 FA | .24 |
| Total PUFA | 58.7 |

TABLE 4
Vitamin E in Red Raspberry Seed Oil

| Total Tocopherol | grams/100 g |
|---|---|
| Alpha Tocopherol | 1.64 mg |
| Beta Tocopherol | <.500 mg |
| Gamma Tocopherol | 6.05 mg |
| Delta Tocopherol | <.500 mg |

TABLE 5
Vitamin E in Black Cumin Seed Oil

| Total Tocopherol | grams/100 g |
|---|---|
| Beta Tocotrienol | 1.22 mg |
| Gamma Tocopherol | 2.59 mg |

### Methods for Tests

Antioxidant Analysis Using a PHOTOCHEM instrument, a method of photochemiluminescence, combines a very fast photochemical radical generation with a highly sensitive luminometric detection. Free radicals, superoxide anion radicals) are produced by optical excitation of a photosensitizer. These radicals are partially eliminated from the sample by reaction with the antioxidants present in the sample. In the measuring cell the remaining radicals cause the detector substance to luminescence. Subsequently the antioxidant capacity of the sample is determined in a separate cell by means of a photomultiplier tube. The results are presented in equivalent in synthetic vitamin E or TROLOX.

Fatty Acid Analysis. One mg of each oil sample was used to prepare the fatty acid methyl esters (FAME) for gas chromatograph (GC) analysis. The GC analysis of the FA composition was performed on a HP® 6890 gas chromatograph equipped with an autosampler, Chemstation and FID (Hewlitt Packard Co., Avondale, Pa.). A fused silica capillary column SP™-2380 (30 m×0.25 mm with a 0.25 μm film thickness) from Supelco (Bellafonte, Pa.) was used with helium as the carrier gas. The following temperature program was used: 165° C. for 20 mm followed by a 5°/Cmin increase to 185° C., which was then held for 10 mm.

In another embodiment the oil can be extracted from the raw plant material using Super Critical Fluid Extraction technology. Super Critical Fluid Extraction uses CO₂ under pressure to enter the cell walls of the plant material to force the separation of oils and extracts. By varying temperature and pressure different separations or fractions can be achieved. Because the process works at low temperature and in the absence of oxygen, the resulting oil is of high quality and unaltered from its natural form.

Both red and black raspberry oil can be used in this invention. It should be mentioned that other natural oils such as those derived from black cumin seed, caneberries (raspberry, blackberry, marionberry, boysenberry and evergreen blackberry), coriander, sea buckthorn, palm fruit oil and cardomon are also known to have anti-inflammatory activity and COX-2 activity. Finally, combinations of the various oils discussed above are also contemplated by the invention and are therefore within its scope.

### DETAILED DESCRIPTION OF THE INVENTION

Summary of results. A number of specialty cold-pressed edible seed oils, flours, and their blends exhibited significant COX-2 inhibitory capacities using an in vitro enzyme assay. These data suggest the potential utilization of these oils, flours, and their combinations as anti-inflammatory agents and for cancer prevention. COX-2 inhibitory effects of the cold-pressed oils are summarized in Table 1, whereas the COX-2 inhibitory activities of the cold-pressed flours are listed in the Table 2.

General procedure of COX-2 inhibitor extraction. 1 g of each specialty cold-pressed edible seed oils was mixed and extracted with 3×3 mL methanol at ambient temperature. After separation, the methanol extractions were combined and kept in dark under nitrogen at ambient temperature until COX-2 inhibition assay. Each gram of the selected flour was extracted with 10 mL of 70% ethanol at ambient temperature for 15 hours. The supernatant was collected.

General procedure of COX-2 inhibitory assay. The COX-2 inhibitory capacities of the oil and flour extracts were estimated using a commercial kit. The tests were done utilizing the Cayman chemical COX Inhibitory Screening Assay (Catalog No. 560131). This Catalog is expressly incorporated herein by reference. Aspirin was used as standard, and the COX-2 inhibitory ability of each oil or flour extract was expressed as aspirin equivalents per gram of oil or flour.

TABLE 1
COX-2 inhibitory activity of the cold-pressed edible seed oils

| Cold-pressed edible | COX-2 inhibitory activity |
|---|---|
| seed oil samples | (aspirin equivalents mg/g) |
| Black raspberry seed oil | 41.1 |
| Evening primrose seed oil | 26.3 |
| Black cumin seed oil | 228.1 |
| Cranberry seed oil | 34.7 |
| Red raspberry seed oil | 207.0 |
| Mullein seed oil | 201.6 |
| Parsley seed oil | 34.8 |
| Carrot seed oil | 108.8 |
| Blueberry seed oil | 49.5 |

TABLE 2
COX-2 inhibitory activity of the cold-pressed edible seed flours*

| Cold-pressed | COX-2 inhibitory activity |
|---|---|
| edible seed flour | (aspirin equivalents mg/g) |
| Black raspberry seed flour | 114.1 |
| Chardonnay grape seed flour | 53.3 |
| Cranberry seed flour | 48.6 |
| Red raspberry seed flour | 66.7 |
| Merlot grape seed flour | 45.7 |
| Blueberry seed flour | 58.9 |

The present invention encompasses pharmaceutical compositions containing the instant seed oil, seed flour and derivatives thereof containing preparations optionally in combination with acceptable pharmaceutical carriers or excipients.

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art, especially in light of the detailed disclosure provided herein.

The amount of composition administered will be dependent upon the condition being treated, the subject being treated, on the subject's weight, the severity of the affliction, the manner of administration and the judgment of the personalizing physician.

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries which facilitate processing of the compositions compounds into preparation which can be used pharmaceutically. Proper formulation is dependent upon the route of administration chosen.

For injection, the agents of the invention may be formulated in aqueous solutions, preferably in physiologically compatible buffers such as Hanks solution, Ringer's solution, or physiological saline buffer. For transmucosal administration, penetrants appropriate to the barrier to be permeated are used in the formulation. Such penetrants are generally known in the art.

For oral administration, the compositions can be formulated readily by combining the active compositions with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained as a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP).

If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as fit, sealed capsules made of gelatin and a plasticizer, such as glycerol or sorbitol. The push-fit capsules can contain the active ingredients in a mixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compositions for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a power mix of the compound and a suitable powder base such as lactose or starch.

The compositions may be formulated for parenteral administration by injection, e.g., by bolus injection or continuous infusion. Formulations for injection may be presented in unit dosage form, e.g., in ampoules or in multidose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active composition may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compositions may also be formulated in rectal compositions such as suppositories or retention enemas, e.g., containing conventional suppository bases such as cocoa butter or other glycerides.

In addition to the formulations described previously, the compositions may also be formulated as a depot preparation. Such long acting formulations may be administered by implantation (for example subcutaneously or intramuscularly) or by intramuscular injection. Thus, for example, the compositions may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable routes of administration may, for example, include oral, rectal, transmucosal, transdermal, or intestinal administration, parenteral delivery, including intramuscular, subcutaneous, intramedullary injections, as well as intrathecal, direct intraventricular, intravenous, intraperitoneal, intranasal, or intraocular injections.

Alternatively, one may administer the composition in a local rather than systemic manner, for example, via injection of the compound directly into an affected area, often in a depot or sustained release formulation.

Furthermore, one may administer the drug in a targeted drug delivery system, for example, in a liposome coated with an antibody specific for affected cells. The liposomes will be targeted to and taken up selectively by the cells.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a composition of the invention formulated in a compatible pharmaceutical carrier may also be prepared, placed in an appropriate container, and labeled for treatment of an indicated condition. Suitable conditions indicated on the label may include treatment of a disease.

### Dietary Supplements

Dietary supplements suitable for use in the present invention include compositions wherein the active ingredients are contained in an effective amount to achieve its intended purpose. More specifically, an effective amount means an amount effective to prevent development of or to alleviate the existing symptoms of the subject being treated. Determination of the effective amounts is well within the capability of those skilled in the art, especially in light of the detailed disclosure provided herein. The amount of composition administered will be dependent upon the condition being treated, the subject being treated, on the subjects weight, the severity of the affliction, the manner of administration and the judgment of the personalizing physician.

The ingredients of the dietary supplement of this invention are contained in acceptable excipients and/or carriers for oral consumption. The actual form of the carrier, and thus, the dietary supplement itself, may not be critical. The carrier may be a liquid, gel, gelcap, capsule, powder, solid tablet (coated or non-coated), tea or the like. Suitable excipient and/or carriers include maltodextrin, calcium carbonate, dicalcium phosphate, tricalcium phosphate, microcrystalline cellulose, dextrose, rice flour, magnesium stearate, stearic acid, croscarmellose sodium, sodium starch glycolate, crospovidone, sucrose, vegetable gums, agar, lactose, methylcellulose, povidone, carboxymethylcellulose, corn starch, and the like (including mixtures thereof). The various ingredients and the excipient and/or carrier are mixed and formed into the desired form using conventional techniques. Dose levels/unit can be adjusted to provide the recommended levels of ingredients per day in a reasonable number of units.

The dietary supplement may also contain optional ingredients including, for example, herbs, vitamins, minerals, enhancers, colorants, sweeteners, flavorants, inert ingredients, and the like. Such optional ingredients may be either naturally occurring or concentrated forms. Selection of one or several of these ingredients is a matter of formulation, design, consumer preference and end-user. The amounts of these ingredients added to the dietary supplements of this invention are readily known to the skilled artisan. Guidance to such amounts can be provided by the U.S. RDA doses for children and adults.

### REFERENCES

Harris, R. E.; Beebe-Donk, J.; Doss, H.; and Doss, D. B. 2005. Aspirin, ibuprofen, and other non-steroidal anti-inflammatory drugs in cancer prevention: A critical review of non-selective COX-2 blockade (review). Oncol Rep. 13, 559-83.
Michaux, C.; and Charlier, C. 2004. Structural approach for COX-2 inhibition. Mini-Reviews in Medicinal Chemisry. 4, 603-615.
Patel, M. I.; Subbaramaiah, K.; Du, B.; Chang, M.; Yang, P.; Newman, R. A.; Cordon-Cardo, C.; Thaler, H. T.; and Dannenberg, A. J. 2005. Celecoxib inhibits prostate cancer growth: evidence of a cyclooxygenase-2-independent mechanism. Clin Cancer Res. 11, 1999-2007.
De Leval, X.; Julemont, F.; Benoit, V.; Frederich, M.; Pirotte, B.; and Dogne, J.-M. 2004. First and second generation of COX-2 selective inhibitors. Mini-Reviews in Medicinal Chemisry. 4, 597-601.
Swamy, M. V.; Cooma, I.; Patlolla, J. M.; Simi, B.; Reddy, B. S.; and Rao, C. V. 2004. Modulation of cyclooxygenase-2 activities by the combined action of celecoxib and decosahexaenoic acid: novel strategies for colon cancer prevention and treatment. Mol Cancer Ther. 3, 215-221.

We claim:

1. An anti-inflammatory and COX-2 inhibitory composition that comprises at least one of cold-pressed edible seed oils, flours and/or their derivatives having anti-inflammatory and COX-2 inhibitory activity as an active ingredient.

2. The anti-inflammatory and COX-2 inhibitory composition of claim 1, wherein the anti-inflammatory and COX-2 inhibitory composition active ingredient is derived from at least one of red raspberry, black raspberry seed, black cumin seed, caneberry seed, raspberry seed, blackberry seed, marionberry seed, boysenberry seed, evergreen blackberry seed, coriander seed, sea buckthorn seed, palm fruit seed, cardomon seed, evening primrose seed, cranberry seed, mullein seed, parsley seed, carrot seed, blueberry seed, grape seed, chardonnay grape seed or merlot grape seed.

3. The anti-inflammatory and COX-2 inhibitory composition of claim 1, wherein the anti-inflammatory composition and COX-2 inhibitory wherein the active ingredient is derived from at least one of red or black raspberry seed or black cumin seed.

4. The anti-inflammatory and COX-2 inhibitory composition of claim 1, wherein the anti-inflammatory composition and COX-2 inhibitory wherein the active ingredient is derived from red raspberry seed and black cumin seed.

5-7. (canceled)

8. An anti-inflammatory and COX-2 inhibitory agent, comprising: an anti-inflammatory and COX-2 inhibitory effective amount of the anti-inflammatory and COX-2 inhibitory composition of any one of claims 1-7 and an acceptable carrier or excipient.

9. An adjuvant agent, comprising: an anti-inflammatory and COX-2 inhibitory effective amount of the anti-inflammatory and COX-2 inhibitory composition of any one of claims 1-7 and an acceptable carrier or excipient.

10. A method of treating or ameliorating a condition or disease in a subject suffering from inflammatory and COX-2 over-expression conditions, comprising providing an effective anti-inflammatory and COX-2 inhibitory amount of a composition of cold-pressed edible seed oil from black cumin seed or a derivative thereof, and administering said composition to said subject.

11. The method of claim 10, wherein the subject is a human.

12. The method of claim 10, wherein the subject is a mammal, reptile, fish, pet, bird, domesticated animal, farm animal, animal or other living organism.

13. A method of treating a subject suffering from inflammatory and COX-2 over-expression conditions, comprising the steps of:
a) producing a cold-pressed edible seed oil from black cumin seed;
b) administering a therapeutically-effective amount of a composition of the cold-pressed edible seed oil or a derivative thereof to the subject; and
c) inhibiting the inflaunnatory and COX-2 over-expression conditions.

14. The method of claim 13, wherein the composition is administered by a route selected from the group consisting of administration by: rectal, transdermal, intestinal, parenteral delivery, intramuscular injection, subcutaneous injection, intramedullary injection, intrathecal injection, direct intraventricular injection, intravenous injection, intraperitoneal injection, intranasal injection, and intraocular injection.

15. The method of claim 13, wherein the composition is administered orally.
